(19) United States
(12) Patent Application Publication
(10) Pub. No.: US 2012/0148670 A1
(43) Pub. Date: Jun. 14, 2012

(54) SENSITIVE POLYMER CAPSULE AND METHOD OF MANUFACTURING THE SAME

(75) Inventors: Ki moon Kim, Pohang-si (KR); Jiyeong Lee, Yeongcheon Si (KR); Dongwoo Kim, Pohang-si (KR); Eunju Kim, Pohang-si (KR); Hyuntae Jung, Pohang-si (KR)

(73) Assignee: POSTECH ACADEMAY-INDUSTRY FOUNDATION, Pohang-si, Kyungsangbuk-do (KR)

(21) Appl. No.: 13/390,843

(22) PCT Filed: Aug. 11, 2010
(86) PCT No.: PCT/KR2010/005256

§ 371 Date: Feb. 16, 2012

(30) Foreign Application Priority Data
Aug. 17, 2009 (KR) 1020090075739

(51) Int. Cl. A61K 9/48; A61K 38/38; A61K 31/56; A61K 38/23; A61K 31/704; C08G 73/06; A61K 38/28; B82Y 5/00; B82Y 30/00; B82Y 40/00

(52) U.S. Cl. 424/451; 528/9; 528/367; 528/368; 514/15.2; 514/178; 514/5.9; 514/11.9; 514/34; 428/402; 977/773; 977/897; 977/906

(57) ABSTRACT

A polymer capsule manufactured by polymerizing a compound represented by Formula 1, or polymerizing the compound of Formula 1 and a compound of Formula 2, wherein a detailed structure of the compounds of Formulae 1 and 2 is presented in the detailed description.

### TECHNICAL FIELD

The present invention relates to a polymer capsule and a method of manufacturing the same, and in particular, to a polymer capsule formed by homogeneous polymerization of cucurbituril derivatives or hard planar molecules, or co-polymerization between cucurbituril derivatives or hard planar molecules and a reactive compound, and a method of manufacturing the same.

### BACKGROUND ART

Polymer capsules are polymer materials having hollow spaces inside them and can encapsulate macromolecules such as proteins or DNAs as well as micro-molecules therein. Thus, polymer capsules have been applied as nanoreactors, drug delivery systems, etc. In particular, unlike liposome or micelle, a polymer capsule having a cross-linkage network retains high stability in vivo, and thus, a study on use of the polymer capsule as a drug carrier is being performed

To use such a polymer capsule as a drug carrier in vivo, the polymer material needs to be formed as having a physically or chemically stable structure in vivo and various sizes and shapes. However, most of all, the polymer capsule should be a biodegradable material that decomposes inside a cell and a biocompatibility polymer material that has low toxicity. The inside of a cell has a weak acidic condition and the cell itself generates glutathione as an antioxidant. Based on such characteristics and various enzymatic functions inside a cell, a study on biodegradable polymer material is actively being performed, and the construction of an effective drug delivery system is critical.

For example, there is a need to develop a drug delivery system that does not decompose in blood, that decomposes in a cell, and that effectively delivers a drug into a cytoplasm.

### DETAILED DESCRIPTION OF THE INVENTION

### Technical Problem

The present invention provides a novel polymer capsule.

The present invention also provides a method of manufacturing a polymer capsule.

According to an aspect of the present invention, there is provided a polymer capsule manufactured by polymerizing a compound represented by Formula 1 below, or polymerizing the compound represented by Formula 1 below and a compound represented by Formula 2 below:

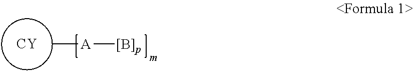

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group are optionally substituted with one or more selected from the group consisting of —(C═O)—, —O(C═O)—, —O—, —S—, and —NH,

a plurality of B are each independently a C1 to 20 alkyl group, a C1 to 20 alkoxy group, a —C(═O)H, —COOH, —CH═CH₂, —C≡CH, —OH, or —NH₂,

-A-(B)p includes one or more molecules other than carbon and hydrogen, and

p is an integer of 1 to 3, and m is an integer of 3 to 23, and

(Y₁)ⱼ—Z—(Y₂)ₖ <Formula 2>

In Formula 2,

Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,

Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and

j and k are each independently an integer of 1 to 3.

According to another aspect of the present invention, there is provided a method of manufacturing a polymer capsule, wherein the method includes mixing a compound represented by Formula 1 below and a compound represented by Formula 2 below:

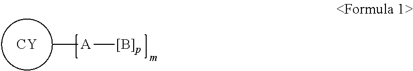

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group are optionally substituted with one or more selected from the group consisting of —(C═O)—, —O(C═O)—, —O—, —S—, and —NH,

a plurality of B are each independently a C1 to 20 alkyl group, a C1 to 20 alkoxy group, —C(═O)H, —COOH, —CH═CH₂, —C≡CH, —OH, or —NH₂,

-A-(B)p includes one or more molecules other than carbon and hydrogen, and

p is an integer of 1 to 3, and m is an integer of 3 to 23.

### Technical Solution

According to an aspect of the present invention, there is provided a polymer capsule manufactured by polymerizing a compound represented by Formula 1 below, or polymerizing the compound represented by Formula 1 below and a compound represented by Formula 2 below:

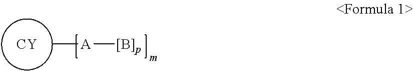

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group may be optionally substituted with one or more selected from the group consisting of —(C═O)—, —O(C═O)—, —O—, —S—, and —NH,

a plurality of B are each independently a C1 to 20 alkyl group, a C1 to 20 alkoxy group, —C(═O)H, —COOH, —CH═CH₂, —C≡CH, —OH, or —NH₂,

-A-(B)p includes one or more molecules other than carbon and hydrogen, and

p is an integer of 1 to 3, and m is an integer of 3 to 23, and

(Y₁)ⱼ—Z—(Y₂)ₖ <Formula 2>

in Formula 2,

Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,

Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and

j and k are each independently an integer of 1 to 3.

According to an aspect of the present invention, there is provided a method of manufacturing a polymer capsule, wherein the method includes mixing a compound represented by Formula 1 below and a reaction catalyst to form a polymer capsule:

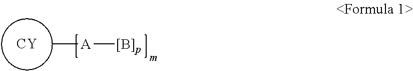

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group may be optionally substituted with one or more selected from the group consisting of —(C═O)—, —O(C═O)—, —O—, —S—, and —NH,

a plurality of B are each independently a C1 to 20 alkyl group, a C1 to 20 alkoxy group, a —C(═O)H, —COOH, —CH═CH₂, —C≡CH, —OH, or —NH₂,

-A-(B)p includes one or more molecules other than carbon and hydrogen, and

p is an integer of 1 to 3, and m is an integer of 3 to 23.

### ADVANTAGEOUS EFFECTS

A polymer capsule according to the present invention decomposes not in blood, but in cells, and due to the inclusion of a pharmaceutically active material and/or target-specific material in the polymer capsule, a drug may be effectively delivered into a cytoplasm.

### DESCRIPTION OF THE DRAWINGS

FIG. 1 is a schematic view of a polymer capsule encapsulating a pharmaceutically effective material, wherein a surface of the polymer capsule is reformed with a target-specific compound;

FIG. 2 is a transmission electron microscope (TEM) image of a polymer capsule prepared according to Example 1;

FIG. 3 is a TEM image of a polymer capsule prepared according to Example 2;

FIG. 4 is a TEM image of a polymer capsule prepared according to Example 3;

FIG. 5 is a TEM image of a polymer capsule prepared according to Example 4;

FIG. 6 is an ultraviolet (UV) absorption graph of a polymer capsule having a disulfide group and encapsulating albumin, prepared according to Example 4;

FIG. 7 is a TEM image of a polymer capsule having a disulfide group and encapsulating hydrocortisone, prepared according to Example 5;

FIG. 8 shows electrospray ionization (ESI)-Mass measurement results of the polymer capsule having a disulfide group and encapsulating hydrocortisone, prepared according to Example 5;

FIG. 9 is a TEM image of a polymer capsule having a disulfide group and encapsulating insulin, prepared according to Example 6;

FIG. 10 is a TEM image of a polymer capsule having a disulfide group and encapsulating calcitonin, prepared according to Example 7;

FIG. 11 is a TEM image of the polymer capsule having a disulfide group and encapsulating doxorubicin, prepared according to Example 8;

FIG. 12 is a UV absorption graph of a polymer capsule having a disulfide group and encapsulating doxorubicin, prepared according to Example 8;

FIG. 13 is a TEM image of a polymer capsule encapsulating doxorubicin and having a disulfide group and including folate-spermidine, prepared according to Example 9;

FIG. 14 is a UV absorption graph of the polymer capsule encapsulating doxorubicin and having a disulfide group and including folate-spermidine, prepared according to Example 9;

FIG. 15 is a TEM image of a polymer capsule having an acetal linkage (polymer capsule prepared according to Example 2), after the polymer capsule was left in a pH of 5.5, according to Example 10;

FIG. 16 a TEM image of a polymer capsule having a disulfide group (polymer capsule prepared according to Example 3) after the polymer capsule was left in a 5 mM glutathione condition according to Example 10;

FIG. 17 is a TEM image of a polymer capsule encapsulating 5(6)-carboxyfluorescein and having a disulfide group, prepared according to Example 11;

FIG. 18 is a graph of fluorescence intensity of a polymer capsule encapsulating 5(6)-carboxyfluorescein and having a disulfide group over time, prepared according to Example 11, when 5 mM glutathione is added to the polymer capsule;

FIG. 19 is a TEM image of a polymer capsule encapsulating 5(6)-carboxyfluorescein and having a disulfide group and folate-spermidine, prepared according to Example 12;

FIG. 20 shows con-focal laser scanning microscope images of (a) KB cells, (c) KB cells treated with a polymer capsule encapsulating 5(6)-carboxyfluorescein and having a disulfide group and included folate-sperimidine, (b) KB cells treated with a polymer capsule encapsulating 5(6)-carboxyfluorescein, having a disulfide group and not having included folate-spermidine, and (d) KB cells treated with a polymer capsule encapsulating 5(6)-carboxyfluorescein, not having a disulfide group and having included folate-spermidine, which are prepared according to Example 12;

FIG. 21 is a graph of viability of KB cells with respect to a doxorubicin concentration, wherein the KB cells were treated with various concentrations of a polymer capsule encapsulating doxorubicin prepared according to Example 9; and

FIG. 22 is a schematic diagram of a target-specific compound included in an inner cavity of a cucurbituril ring.

### BEST MODE

Hereinafter, a polymer capsule according to the present invention and a method of manufacturing a polymer capsule are described in detail.

A polymer capsule according to the present invention may be prepared by polymerizing a compound represented by Formula 1 below, or polymerizing the compound represented by Formula 1 below and a compound represented by Formula 2 below:

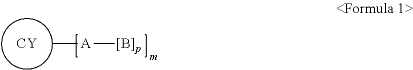

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group may be optionally substituted with one or more selected from the group consisting of —(C═O)—, —O(C═O)—, —O—, —S—, and —NH,

a plurality of B are each independently a C1 to 20 alkyl group, a C1 to 20 alkoxy group, a —C(═O)H, —COOH, —CH═CH₂, —C≡CH, —OH, or —NH₂,

-A-(B)p further includes one or more molecules other than carbon and hydrogen, and

p is an integer of 1 to 3, and m is an integer of 3 to 23,

(Y₁)ⱼ—Z—(Y₂)ₖ <Formula 2>

In Formula 2,

Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,

Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and

j and k are each independently an integer of 1 to 3.

Otherwise defined herein, an aromatic ring refers to a carbocyclic aromatic ring including carbon atoms as ring atoms.

Otherwise defined herein, a heteroaromatic ring refers to an aromatic ring that includes one, two, or three hetero atoms selected from nitrogen (N), oxygen (O), and sulfur (S) and a carbon atom as the residual ring atom.

The compound represented by Formula 1 may be polymerized to form a polymer capsule in the presence of an appropriate reaction catalyst. Alternatively, the compound represented by Formula 1 and the compound represented by Formula 2 may be co-polymerized to form a polymer capsule in which the compounds of Formula 1 are cross-linked via the compound of Formula 2. The polymer capsule may have a hollow therein, so that, for example, a pharmaceutically active material may be encapsulated therein.

The CY may be, for example, a cucurbituril ring or a derivative thereof, a benzene ring or a derivative thereof, a naphthalene ring or a derivative thereof, an anthracene ring or a derivative thereof, a triphenylene ring or a derivative thereof, a pyrene ring or a derivative thereof, a coronin ring or a derivative thereof, a triazine ring or a derivative thereof, a phthalocyanine ring or a derivative thereof, a porphyrin ring or a derivative thereof, a pyridine ring or a derivative thereof, a quinoline ring or a derivative thereof, an anthraquinone ring or a derivative thereof, or a phenanthroline ring or a derivative thereof, but is not limited thereto, and any other cucurbituril ring derivatives that are known in the art and a hard planar aromatic ring may also be used as CY.

-A-(B)p may be, for example, a hydroxy group, a methyloxy group, a butyloxy group, a dodecabutyloxy group, a propynyloxy group, a hydroxyethyloxy group, a C1 to 30 alkyloxycarbonyloxy group, a C1 to 30 alkylcarbonyloxy group, a C1 to 30 aminoalkyloxy group, —OC(═O)CH₃, —OCH₂CH₂CH₂SCH₂COOH, —OCH₂CH₂CH₂SCH₂CH₂NH₂, or —OC(═O)CH₂CH₂CH₂CH₂CH₂CH₂CH₂CH₂CH₂CH₂CH₃.

Also, various polymer capsules may be formed by combining the compound of Formula 1 and the compound of Formula 2.

For example, in the compound of Formula 1 having 3 to 20 double bonds or triple bonds (—CH═CH₂, —C≡CH) at its terminal, the terminal double bonds are linked to each other by olefin cross-metathesis to form a polymer capsule.

Also, the compound of Formula 1 having 3 to 20 hydroxy groups may react with the compound of Formula 2 having 2 or more vinyloxy groups under an acidic catalytic condition to form a polymer capsule that has a polyacetal crosslinking. The polyacetal linking decomposes under an acidic condition, which enables the formation of a biodegradable polymer capsule.

Also, the compound of Formula 1 having 3 to 20 amino groups may react with the compound of Formula 2 having 2 or more N-acetoxysuccinimide groups under a basic catalytic condition to form a disulfide group, thereby forming a polymer capsule. Also, if Z included in the compound of Formula 2 having 2 or more N-acetoxysuccinimide groups includes a disulfide group, it may be a decomposable one due to oxidation and reduction, thereby enabling the formation of a biodegradable polymer capsule as an effective drug delivery system.

Regarding the polymer capsule, the compound represented by Formula 1 may be represented by one of Formulae 3 to 12 below:

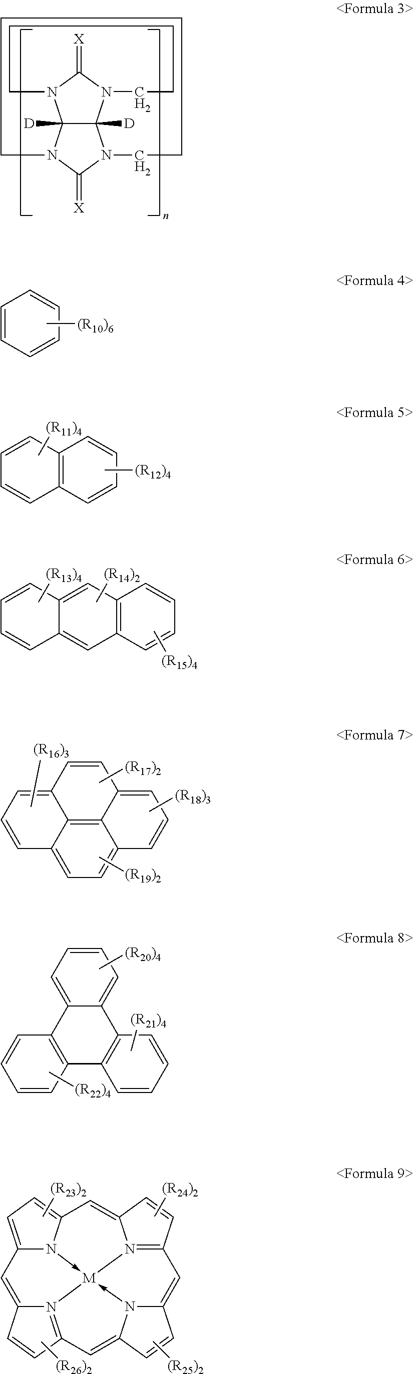

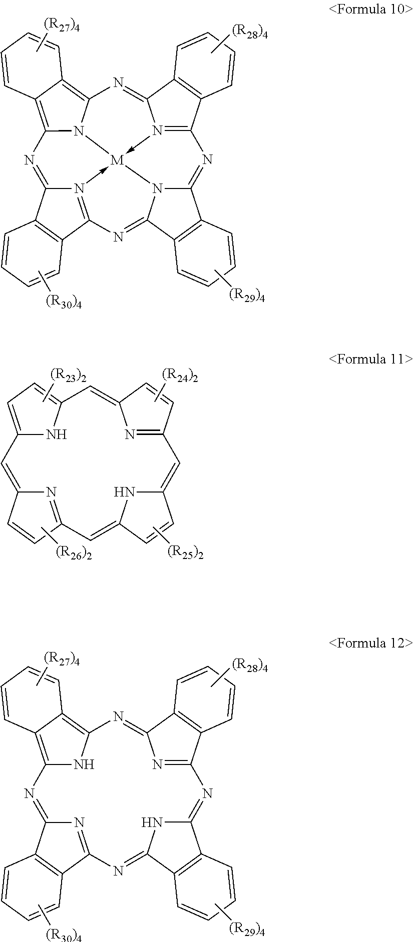

In the formulae above,

a plurality of D are each independently hydrogen or -A-(B)p, and, 3 or more of D are -A-(B)p, a plurality of X are each independently O, S, or NH, and n is an integer of 4 to 20,

R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, and R₃₀ are each independently hydrogen or -A-(B)p, and

3 or more of R₁₀, 3 or more selected from a plurality of R₁₁ and R₁₂, 3 or more selected from a plurality of R₁₃, R₁₄ and R₁₅, 3 or more selected from a plurality of R₁₆, R₁₇, R₁₈ and R₁₉, 3 or more selected from a plurality of R₂₀, R₂₁ and R₂₂, 3 or more selected from a plurality of R₂₃, R₂₄, R₂₅ and R₂₆, and 3 or more selected from a plurality of R₂₇, R₂₈, R₂₉ and R₃₀ are -A-(B)p.

According to another embodiment of the present invention, the polymer capsule may further include a target-specific compound that is included in an inner cavity of the cucurbituril ring. The target-specific compound is a compound that has a functional group having high affinity with a target cell. The target-specific compound may be included near a surface of the polymer capsule in the inner cavity of the polymer capsule to aid the polymer capsule to approach the target cell. The target-specific compound may be represented by Formula 11 below.

E₁-G-E₂ <Formula 11>

In Formula 10,

G is a chemical bond, a C1 to 30 alkylene group, a C2 to 30 alkenylene group, a C2 to 30 alkynylene group, a C5 to 30 cycloalkylene group, a C6 to 30 arylene group, a C2 to 30 heteroarylene group, a C7 to 30 alkylarylene group, or a C7 to 30 arylalkylene group,

one or more carbon atoms of the alkylene group, alkenylene group, alkynylene group, cycloalkylene group, arylene group, or heteroarylene group may be optionally substituted with one or more selected from the group consisting of —Si(Ra)(Rb)- (where Ra and Rb are each independently a C1 to 10 alkyl group), —(C═O)—, —O(C═O)—, —O—, —S—, and —NH—,

E₁ is a 1,3-diaminopropyl group, a 1,4-diaminobutyl group, a 1,5-diaminopentyl group, a 1,6-diaminohexyl group, a sperminyl group, a spermidinyl group, a propylamino group, a butylamino group, a pentylamino group, a hexylamino group, a viologenyl group, a pyridinyl group, a ferrocenyl group, or an amino acid group, and

E₂ is a radical of sugar, polypeptide, a protein, or a gene from which one hydrogen atom is removed, or a cation of sugar, polypeptide, a protein, or a gene from which one electron is removed.

The sugar, that is one of the target-specific material, may be, for example, glucose, mannose, or galactose, but is not limited thereto, and any other sugar that is known in the art may also be used as the sugar herein.

The protein, that is one of the target-specific material, may be, for example, lectin, selectin, or transpherine, but is not limited thereto, and any other protein that is known in the art may also be used as the protein herein.

For example, the target-specific compound may be folate-spermidine, glucose-sperminidine, mannose-sperminidine, galactose-sperminidine, lectin-spermine, cellectin-spermine, transferrin-spermine, or a combination thereof.

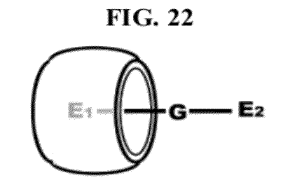

FIG. 22 is a schematic view of the target-specific compound included in an inner cavity of the cucurbituril ring. Referring to FIG. 22, the oval rings indicate cucurbituril rings. Referring to FIG. 22, since a portion E₁ of the target-specific compound is designed using a substituent that is well included in the cucurbituril derivative, the portion E₁ is well included in the inner cavity of the cucurbituril derivative that is exposed to the surface of a polymer capsule formed from the cucurbituril derivative. Accordingly, as illustrated in FIG. 22, the surface of the polymer capsule is reformable with E₂ that is connected to E₁ via G, wherein E₂ is a target-specific substituent and G is a linking portion.

According to another embodiment of the present invention, the polymer capsule may further include a pharmaceutically active material or a mono-molecular compound encapsulated in the polymer capsule. Due to the inclusion of the pharmaceutically effective material, the polymer capsule may be used as a carrier or a nano-reaction vessel for a pharmaceutically active material, and may function as a drug delivery function.

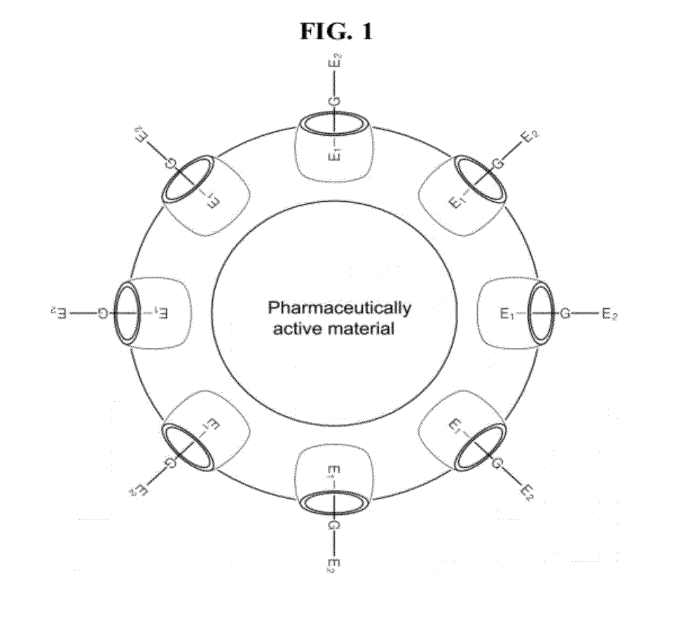

Also, a polymer capsule with the target-specific compound included in its surface may also function as a carrier for the pharmaceutically active material. In particular, if a drug is encapsulated in a polymer capsule that uses cucurbituril and includes the target-specific compound, the drug may function specifically to only a target site in vivo. Thus, side effects that occur in other areas than the target area by action of the drug may be prevented. FIG. 1 is a schematic view of a polymer capsule encapsulating a pharmaceutically effective material, wherein a surface of the polymer capsule is reformed with the compound of Formula 3 as the target-specific compound.

The pharmaceutically active material encapsulated in the polymer capsule may not be limited to a particular material, and may be one of various materials that have pharmaceutical activity and can be dissolved or dispersed in a solvent used in preparing the polymer capsule. For example, the pharmaceutically active material may be an organic compound, a protein, a gene, or the like.

The pharmaceutically active material may be, for example, hydrocortisone, predsisolone, spironolactone, testosterone, megestrol acetate, danazol, progesterone, indomethacin, amphotericin B, or a combination thereof.

The pharmaceutically active material may be, for example, a human growth hormone, a G-CSF, a granulocyte-macrophage colony-stimulating factor (GM-CSF), erythropoietin, vaccine, an antibody, insulin, glucagon, calcitonin, an adrenocorticotropic hormone (ACTH), somatostatin, somatotropin, somatomedin, a parathyroid hormone, a thyroid hormone, a hypothalamic secretion material, prolactin, endorphin, a vascular endothelial growth factor (VEGF), enkephalin, vasopressin, a nerve growth factor, a non-naturally occurring opioid, interferon, asparaginase, alginase, superoxide dismutase, trypsin, chymotrypsin, pepsin, or a combination thereof.

According to an embodiment of the present invention, a diameter of the polymer capsule may be in a range of 10 to 9000 nm. For example, the diameter of the polymer capsule may be in a range of 10 to 5000 nm.

A method of manufacturing the polymer capsule according to an embodiment of the present invention includes mixing the compound represented by Formula 1 below with a reaction catalyst to form a polymer capsule:

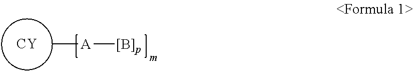

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group may be substituted with one or more selected from the group consisting of —(C═O)—, —O(C═O)—, —O—, —S—, and —NH, a plurality of B are each independently a C1 to 20 alkyl group, a C1 to 20 alkoxy group, a —C(═O)H, —COOH, —CH═CH₂, —C≡CH, —OH, or —NH₂,

-A-(B)p includes one or more molecules other than carbon and hydrogen, and

p is an integer of 1 to 3, and m is an integer of 3 to 23.

For example, the compound represented by Formula 1 is mixed with a reaction catalyst and the mixture is dissolved in a solvent, followed by 1 to 100 hours of mixing to proceed a polymerization reaction of the compound represented by Formula 1. When the polymerization reaction is finished, the non-reacted compound represented by Formula 1 and the non-reacted reaction catalyst are removed by filtering, thereby completing the preparation of the polymer capsule.

The reaction catalyst used in this method may be a Grubbs catalyst, an acidic catalyst, a basic catalyst, or a combination thereof. The acidic catalyst may be para-toluene sulfonate, para-toluenesulfonyl chloride, HCl, H₂SO₄, HNO₃, or a combination thereof, and the basic catalyst may be N(CH₂CH₃)₃, pyridine, NaOH, NaBH₄, LiAlH₄ or a combination thereto. However, the acidic and basic catalysts are not limited thereto, and may each be one of various catalysts that are used in the art and induce a reaction of a reactive terminal group included in the compound represented by Formula 1.

According to an embodiment of the present invention, a diameter of a polymer capsule manufactured by using the method may be in a range of 10 to 9000 nm. For example, the diameter of the polymer capsule may be in a range of 10 to 5000 nm.

A method of manufacturing the polymer capsule according to another embodiment of the present invention includes mixing the compound represented by Formula 1 and the compound represented by Formula 2 to form a polymer capsule:

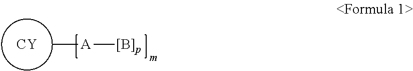

In Formula 1,

CY is a cucurbituril ring, a C2-C50 heteroaromatic ring, or a C6-C50 aromatic ring,

a plurality of A are each independently a chemical bond, or a C1 to 20 alkylene group, wherein one or more carbon atoms of the alkylene group may be substituted with one or more selected from the group consisting of —(C═O)—, —O(C═O)—, —O—, —S—, and —NH,

a plurality of B are each independently a C1 to 20 alkyl group, a C1 to 20 alkoxy group, —C(═O)H, —COOH, —CH═CH₂, —C≡CH, —OH, or —NH₂,

-A-(B)p includes one or more molecules other than carbon and hydrogen, and

p is an integer of 1 to 3, and m is an integer of 3 to 23,

(Y₁)ⱼ—Z—(Y₂)ₖ <Formula 2>

In Formula 2,

Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,

Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and

j and k are each independently an integer of 1 to 3.

According to an embodiment of the present invention, a diameter of a polymer capsule manufactured by using the method according to the present embodiment may be in a range of 10 to 9000 nm. For example, the diameter of the polymer capsule may be in a range of 10 to 5000 nm.

For example, when the compounds of Formulae 1 and 2 are mixed and the mixture is dissolved in a solvent, followed by 1 to 100 hours of mixing, copolymerization may be performed between the compounds of Formulae 1 and 2. When the copolymerization is finished, the non-reacted compounds of Formulae 1 and 2 are removed by filtering, thereby completing the preparation of a polymer capsule.

According to another embodiment of the present invention, the mixing of the compound represented by Formula 1 and the compound represented by Formula 2 to form a polymer capsule, and/or the mixing the compound represented by Formula 1 and the compound represented by Formula 2 to form a polymer capsule may be performed additionally using a pharmaceutically active material. Due to the use of the pharmaceutically active material in forming a polymer capsule, the pharmaceutically active material may be encapsulated in the polymer capsule.

For example, the compound represented by Formula 1, a pharmaceutically active material, and a reaction catalyst may be mixed to form a polymer capsule. For example, the compound represented by Formula 1, the compound represented by Formula 2, and a pharmaceutically active material are mixed to form a polymer capsule that encapsulates a pharmaceutically active material.

The pharmaceutically active material may not be limited to a particular material, and may be one of various materials that have pharmaceutical activity and can be dissolved or dispersed in a solvent used in preparing the polymer capsule. For example, the pharmaceutically active material may be an organic compound, a protein, a gene, or the like.

The pharmaceutically active material used in manufacturing the polymer capsule may be, for example, hydrocortisone, predsisolone, spironolactone, testosterone, megestrol acetate, danazol, progesterone, indomethacin, amphotericin B, or a combination thereof,

Also, the pharmaceutically active material may be, for example, a human growth hormone, a G-CSF, a granulocyte-macrophage colony-stimulating factor (GM-CSF), erythropoietin, vaccine, an antibody, insulin, glucagon, calcitonin, an ACTH, somatostatin, somatotropin, somatomedin, a parathyroid hormone, a thyroid hormone, a hypothalamic secretion material, prolactin, endorphin, a VEGF, enkephalin, vasopressin, a nerve growth factor, a non-naturally occurring opioid, interferon, asparaginase, alginase, superoxide dismutase, trypsin, chymotrypsin, pepsin, or a combination thereof.

According to an embodiment of the present invention, a diameter of a polymer capsule prepared by using the method according to the present embodiment may be in a range of 10 to 9000 nm. For example, the diameter of the polymer capsule may be in a range of 10 to 5000 nm.

For example, when the compounds of Formulae 1 and 2 and the pharmaceutically active material are mixed and the mixture is dissolved in a solvent, followed by 1 to 100 hours of mixing, copolymerization may be performed between the compounds of Formulae 1 and 2. When the copolymerization is finished, the non-reacted compounds of Formulae 1 and 2 and the pharmaceutically active material that is not encapsulated in the formed polymer capsule are removed by filtering, thereby completing the preparation of a polymer capsule that encapsulates the pharmaceutically active material.

A method of manufacturing a polymer capsule, according to an embodiment of the present invention, further includes, after the polymer capsule that encapsulates the pharmaceutically active material is formed, mixing the polymer capsule encapsulating the pharmaceutically active material with a target-specific compound to include the target-specific compound in inner cavities of one or more cucurbituril rings that constitute the polymer capsule.

The polymer capsule having the target-specific compound included in its surface is more suitable for use as a carrier for the pharmaceutically effective material. For example, when a drug is encapsulated in a polymer capsule that uses cucurbituril and includes the target-specific compound, the drug may function specifically to only a target site in vivo. Thus, side effects that occur in other areas than the target area by action of the drug may be prevented.

A method of manufacturing a polymer capsule, according to another embodiment of the present invention, includes mixing a compound represented by Formula 3 below, the compound represented by Formula 2, and a pharmaceutically active material to form a polymer capsule that encapsulates the pharmaceutically effective material; and mixing the polymer capsule encapsulating the pharmaceutically active material with a target-specific compound to include the target-specific compound in inner cavities of one or more cucurbituril rings that constitute the polymer capsule.

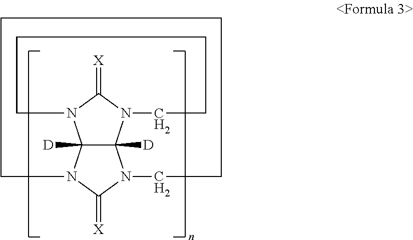

in Formula 3,

a plurality of D are each independently hydrogen or -A-(B)p, a plurality of X are each independently O, S, or NH, n is an integer of 4 to 20, from among D, 3 or more are -A-(B)p, a plurality of A are each independently a chemical bond or a C1 to 20 alkylene group, one or more carbon atoms of the alkylene group may be substituted with one or more selected from the group consisting of —(C═O)—, —O(C═O)—, —O—, —S—, and —NH—, a plurality of B are each independently a C1 to 20 alkyl group, a C1 to 20 alkoxy group, —C(═O)H, —COOH, —CH═CH₂, —C≡CH, —OH, or —NH₂, -A-(B)p includes one or more molecules other than carbon and hydrogen, and p is an integer of 1 to 3, and

(Y₁)ⱼ—Z—(Y₂)ₖ <Formula 2>

In Formula 2,

Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,

Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and

j and k are each independently an integer of 1 to 3.

The pharmaceutically active material used in manufacturing the polymer capsule may be, for example, hydrocortisone, predsisolone, spironolactone, testosterone, megestrol acetate, danazol, progesterone, indomethacin, amphotericin B, or a combination thereof.

Also, the pharmaceutically active material used in manufacturing the polymer capsule may be, for example, a human growth hormone, a G-CSF, a granulocyte-macrophage colony-stimulating factor (GM-CSF), erythropoietin, vaccine, an antibody, insulin, glucagon, calcitonin, an ACTH, somatostatin, somatotropin, somatomedin, a parathyroid hormone, a thyroid hormone, a hypothalamic secretion material, prolactin, endorphin, a VEGF, enkephalin, vasopressin, a nerve growth factor, a non-naturally occurring opioid, interferon, asparaginase, alginase, superoxide dismutase, trypsin, chymotrypsin, pepsin, or a combination thereof.

The target-specific compound used in manufacturing the polymer capsule may be represented by Formula 11 below:

E₁-G-E₂ <Formula 11>

In Formula 10,

G is a chemical bond, a C1 to 30 alkylene group, a C2 to 30 alkenylene group, a C2 to 30 alkynylene group, a C5 to 30 cycloalkylene group, a C6 to 30 arylene group, a C2 to 30 heteroarylene group, a C7 to 30 alkylarylene group, or a C7 to 30 arylalkylene group,

one or more carbon atoms of the alkylene group, alkenylene group, alkynylene group, cycloalkylene group, arylene group, and heteroarylene group may be substituted with one or more selected from the group consisting of —Si(Ra)(Rb)- (where Ra and Rb are each independently a C1 to 10 alkyl group), —(C═O)—, —O(C═O)—, —O—, —S—, and —NH—,

E₁ is a 1,3-diaminopropyl group, a 1,4-diaminobutyl group, a 1,5-diaminopentyl group, a 1,6-diaminohexyl group, a sperminyl group, a spermidinyl group, a propylamino group, a butylamino group, a pentylamino group, a hexylamino group, a biologinyl group, a pyridinyl group, a ferrocenyl group, or an amino acid group, and

E₂ is a radical of sugar, polypeptide, a protein, or a gene from which one hydrogen atom is removed, or a cation of sugar, polypeptide, a protein, or a gene from which one electron is removed.

According to an embodiment of the present invention, a diameter of the polymer capsule formed by using the method according to the present embodiment may be in a range of 10 to 9000 nm. For example, the diameter of the polymer capsule may be in a range of 10 to 5000 nm.

A polymer capsule with the target-specific compound included in its surface may also function as a carrier for a pharmaceutically effective material. In particular, if a drug is encapsulated in a polymer capsule that uses the cucurbituril represented by

Formula 3 and includes the target-specific compound, the drug may function specifically to only a target site in vivo. Thus, side effects that occur in other areas than the target area by action of the drug may be prevented. FIG. 1 is a schematic view of a polymer capsule encapsulating a pharmaceutically effective material, wherein a surface of the polymer capsule is reformed with the compound of Formula 3 as the target-specific compound.

In the methods of manufacturing a polymer capsule, according to the above embodiments of the present invention, the compound of Formula 1, the compound of Formula 2, the compound of Formula 11, and/or the pharmaceutically active material are used as being dissolved in a solvent. The dissolution sequence of these compounds is not limited.

The solvent used in the methods of manufacturing a polymer capsule, according to the above embodiments of the present invention, may be a solvent that dissolves the compound of Formula 1, the compound of Formula 2, the compound of Formula 11, and/or the pharmaceutically effective material, and the solvent may be water, chloroform, methyl alcohol, ethyl alcohol, dimethylsulfoxide, dichloromethane, dimethylformamide, tetrahydrofurane, acetone, acetonitrile, or a combination thereof, but is not limited thereto. An amount of the used solvent may not be limited as long as the compounds are sufficiently dissolved.

### MODE OF THE INVENTION

Hereinafter, the present invention will be described in detail with reference to examples. However, the examples are described for illustrative purposes only and the present invention is not limited to the examples.

### EXAMPLE

### Example 1

### Preparation of Polymer Capsule Through Olefin Cross-Metathesis

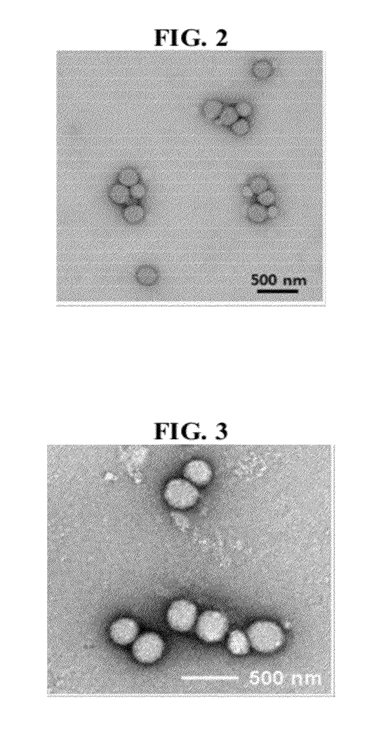

2.7 mg of octapentenyl zinc-phthalocyanine was completely dissolved in about 10 ml of an ethyl alcohol/dimethyl sulfoxide mixed solvent (2:8 volumetric ratio), and then 1.33 mg of a 2nd-generation Grubbs catalyst (Aldrich, No. 569747) was added thereto and dissolved. After 24 hours of stirring, the residual octapentenyl zinc-phthalocyanine and 2nd-generation Grubbs catalyst which were not reacted were removed by dialysis, thereby obtaining a product solution. One droplet of the product solution was dropped onto a planar substrate and dried and a polymer capsule included therein was identified by using a transmission electron microscope. As a result, it was confirmed that a particle size of the polymer capsule was 200 nm and an image thereof is shown in FIG. 2.

### Example 2

### Preparation of Polymer Capsule Through Acetal Linkage

23.8 mg of hydroxycucurbituril[6] was completely dissolved in 10 ml of a dimethyl sulfoxide solution, and 13.7 mg of a para-toluene sulfonic acid and 194.5 mg of triethyleneglycol divinyl ether were added thereto and dissolved. After 24 hours of stirring at room temperature, the residual reactants which were not polymerized were removed by dialysis, thereby obtaining a product solution. One droplet of the product solution was dropped onto a planar substrate and dried and a polymer capsule included therein was identified by using a transmission electron microscope. As a result, it was confirmed that a particle size of the polymer capsule was 70 nm and an image thereof is shown in FIG. 3.

### Example 3

### Preparation of Polymer Capsule Having Disulfide Group Through Polyamide Bonding

3.4 mg (3-(2-aminoethanethio)propane-1-oxy) cucurbituril[6] was completely dissolved in 10 ml of a mixed solution including chloroform and methanol (a volumetric ratio of 5:5), and 50 μl of triethylamine and 2.4 mg of 3-[2-(2,5-dioxo-pyrrolidine-1-siloxycarbonyl)-ethyldisulfanyl]-propionic acid 2,5-dioxo-pyrrolidine-1-cyl ester were added thereto and dissolved. After 24 hours of stirring at room temperature, the residual reactants which were not polymerized were removed by dialysis, thereby obtaining a product solution.

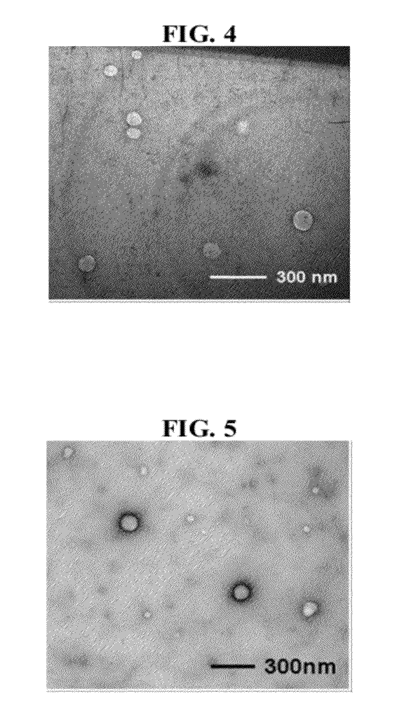

One droplet of the product solution was dropped onto a planar substrate and dried and a polymer capsule included therein was identified by using a transmission electron microscope. As a result, it was confirmed that a particle size of the polymer capsule was 70 nm and an image thereof is shown in FIG. 4.

### Example 4

### Preparation of Polymer Capsule that Encapsulates Albumin (Protein) and has Disulfide Group

3.4 mg (3-(2-aminoethanethio)propane-1-oxy) cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 5 mg of albumin were added thereto and dissolved. After 24 hours of stirring at room temperature, the residual reactants which were not polymerized were removed by dialysis, thereby obtaining a product solution.

One droplet of the product solution was dropped onto a planar substrate and dried and a polymer capsule included therein was identified by using a transmission electron microscope. As a result, it was confirmed that a particle size of the polymer capsule was 90 nm and an image thereof is shown in FIG. 5.

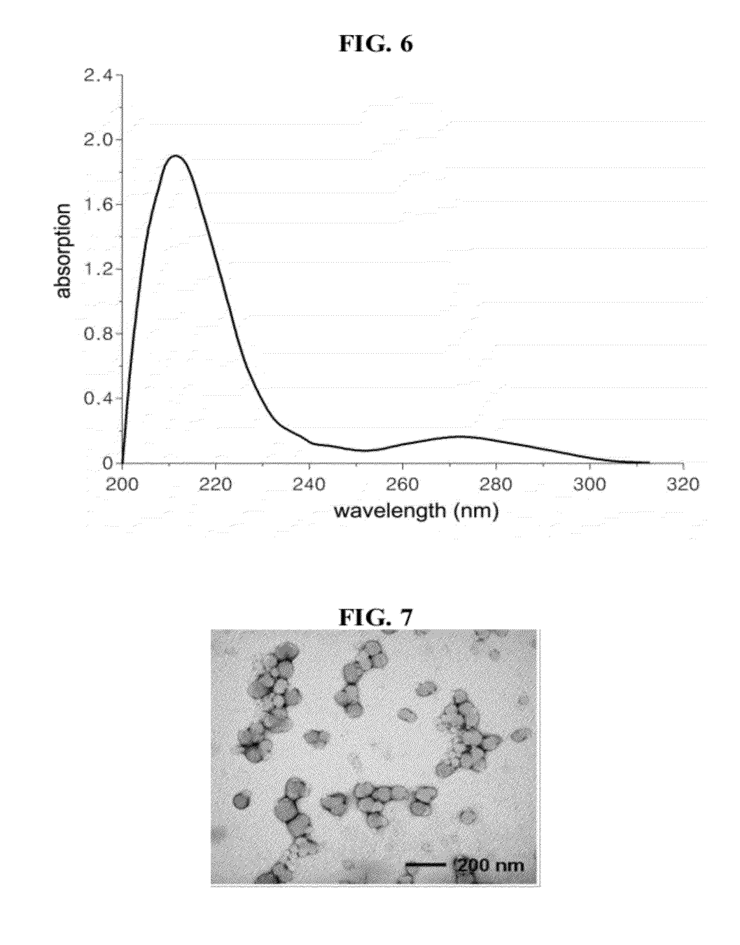

Also, UV-absorption of the polymer capsule prepared according to Example 4 was measured and a strong absorption peak was observed at a wavelength of 280 nm, which is a unique wavelength of albumin. Thus, it was confirmed that albumin was included in the polymer capsule. FIG. 6 shows the UV absorption graph. From these results, it was confirmed that a protein that is smaller than the polymer capsule is sufficiently encapsulated in the polymer capsule.

### Example 5

### Preparation of Polymer Capsule that Encapsulates Hydrocortisone (Organic Compound) and has Disulfide Group

3.4 mg (3-(2-aminoethanethio)propane-1-oxy) cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 1 mg of hydrocortisone were added thereto and dissolved. After 24 hours of stirring at room temperature, the residual reactants which were not polymerized were removed by dialysis, thereby obtaining a product solution.

One droplet of the product solution was dropped onto a planar substrate and dried and a polymer capsule included therein was identified by using a transmission electron microscope. As a result, it was confirmed that a particle size of the polymer capsule was 100 nm and an image thereof is shown in FIG. 7.

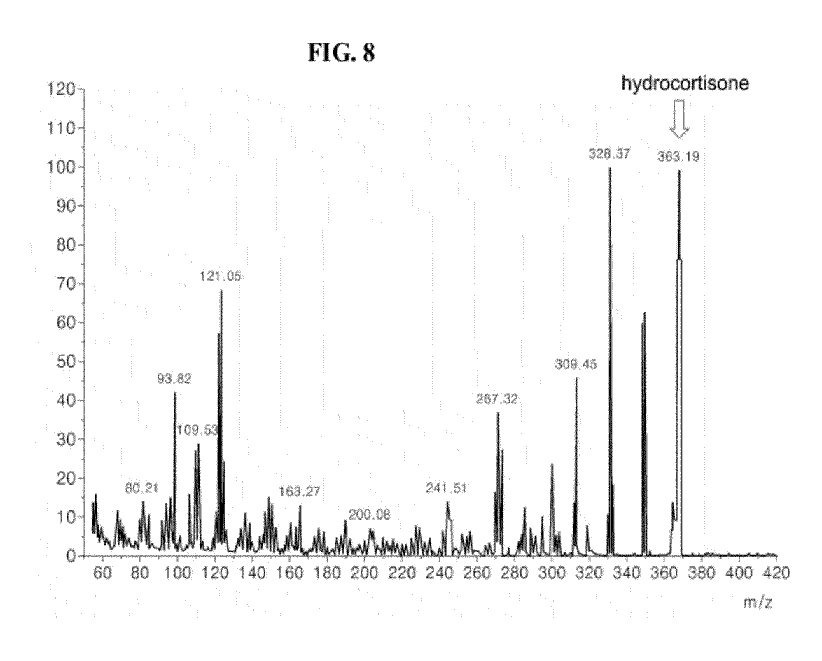

Also, ESI-Mass of the polymer capsule was measured, and due to the presence of a hydrocortisone peak, it was confirmed that hydrocortisone is included in the polymer capsule. ESI-Mass measurement results are shown in FIG. 8. From these results, it was confirmed that an organic compound is sufficiently encapsulated in the polymer capsule.

### Example 6

### Preparation of Polymer Capsule that Encapsulates Insulin and has Disulfide Group

3.4 mg (3-(2-aminoethanethio)propane-1-oxy)cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 1 mg of insulin were added thereto and dissolved. After 24 hours of stirring at room temperature, the residual reactants which were not polymerized were removed by dialysis, thereby obtaining a product solution.

One droplet of the product solution was dropped onto a planar substrate and dried and a polymer capsule included therein was identified by using a transmission electron microscope. As a result, it was confirmed that a particle size of the polymer capsule was 90 nm and an image thereof is shown in FIG. 9.

### Example 7

### Preparation of Polymer Capsule that Encapsulates Calcitonin and has Disulfide Group

3.4 mg (3-(2-aminoethanethio)propane-1-oxy)cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 2 mg of calcitonin were added thereto and dissolved. After 1 day of stirring at room temperature, the residual reactants which were not polymerized were removed by dialysis, thereby obtaining a product solution.

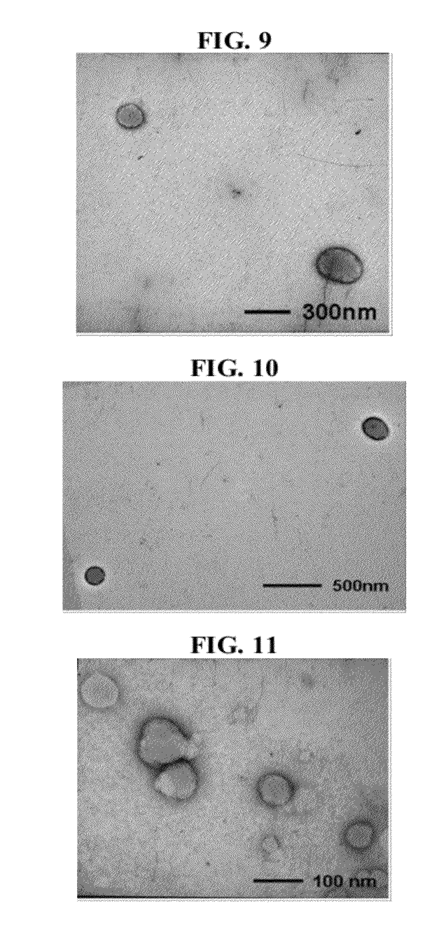

One droplet of the product solution was dropped onto a planar substrate and dried and a polymer capsule included therein was identified by using a transmission electron microscope. As a result, it was confirmed that a particle size of the polymer capsule was 100 nm, and an image thereof is shown in FIG. 10. Also, IR of the polymer capsule was measured, and an amide bond peak corresponding to a strong peptide bond was observed at a wavelength of about 1660 nm. From these results, it was confirmed that calcitonin is sufficiently encapsulated in the polymer capsule.

### Example 8

### Preparation of Polymer Capsule that Encapsulates Doxorubicin and has Disulfide Group

3.4 mg (3-(2-aminoethanethio)propane-1-oxy)cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 2 mg of doxorubicin were added thereto and dissolved. After 24 hours of stirring at room temperature, the residual reactants which were not polymerized were removed by dialysis, thereby obtaining a product solution.

One droplet of the product solution was dropped onto a planar substrate and dried, and a polymer capsule included therein was identified by using a transmission electron microscope. As a result, it was confirmed that a particle size of the polymer capsule was 100 nm and an image thereof is shown in FIG. 11.

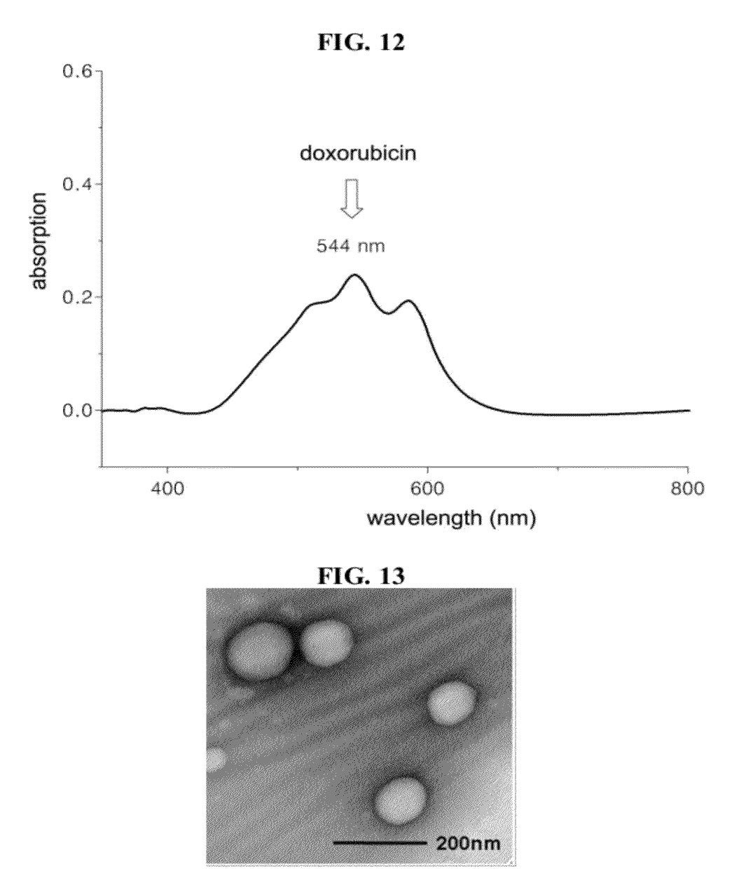

Also, UV-absorption of the polymer capsule prepared according to Example 8 was measured and a strong absorption peak was observed at a wavelength of 544 nm, which is a unique wavelength of doxorubicin. Thus, it was confirmed that albumin was included in the polymer capsule. FIG. 12 shows the UV absorption graph. From these results, it was confirmed that doxorubicin is sufficiently encapsulated in the polymer capsule.

### Example 9

### Preparation of Polymer Capsule that Encapsulates Doxorubicin and has Disulfide Group and Includes Folate-Sperimidine

3.4 mg (3-(2-aminoethanethio)propane-1-oxy) cucurbituril[6] was completely dissolved in 10 ml of a mixed solution including chloroform and methanol (a volumetric ratio of 5:5), and 50 μl of triethylamine, 2.4 mg of 342-(2,5-dioxo-pyrrolidine-1-siloxycarbonyl)-ethyldisulfanyl]-propionic acid 2,5-dioxo-pyrrolidine-1-cyl ester, and 2 mg of doxorubicin were added thereto and dissolved. The result mixture was stirred for 24 hours at room temperature to form a polymer capsule. 0.5 mg of folate-spermidine was added to the reaction solution including the polymer capsule and then stirred for one hour. Then, reactants that were not polymerized, encapsulated, or included were removed by dialysis to obtain a product solution.

One droplet of the product solution was dropped onto a planar substrate and dried, and a polymer capsule included therein was identified by using a transmission electron microscope. As a result, it was confirmed that a particle size of the polymer capsule was 90 nm and an image thereof is shown in FIG. 13.

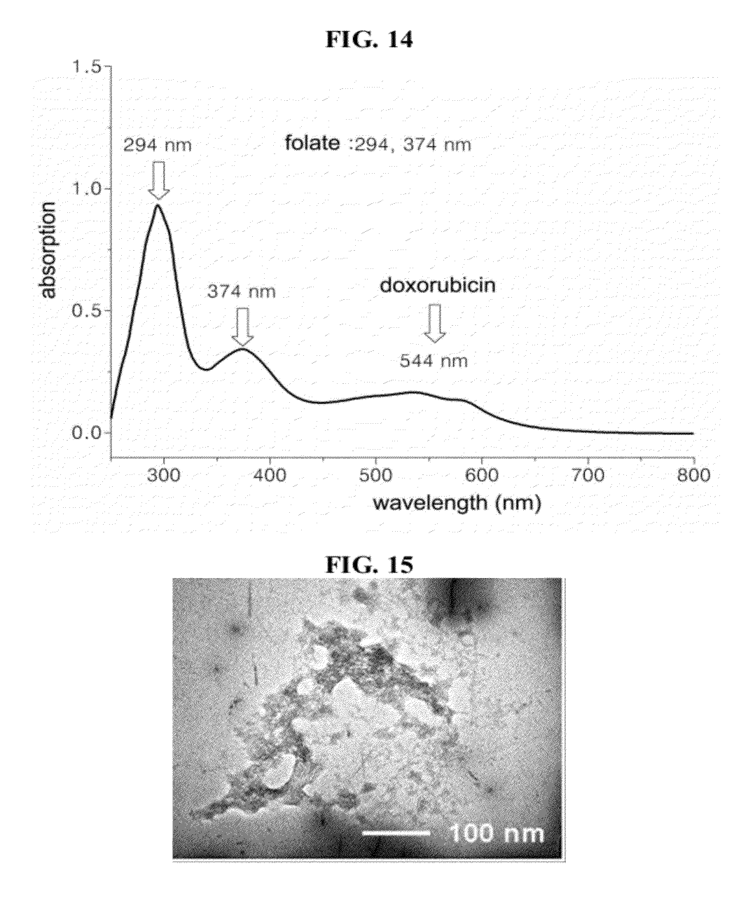

Also, UV-absorption of the polymer capsule prepared according to Example 9 was measured, and the obtained UV absorption graph showed a folate peak corresponding to wavelengths of about 294 and 374 nm and a doxorubicine peak corresponding to a wavelength of 540 nm. FIG. 14 shows the UV absorption graph. From these results, it was confirmed that doxorubicin was encapsulated inside the polymer capsule and folate-spermidine was included outside the polymer capsule.

### Example 10

### Decomposition Test of Polymer Capsule Under Acidic Conditions or Reducing Conditions

The polymer capsule having an acetal linkage prepared according to Example 2 was added to 0.5 N HCl to control a pH to be 5.5, and then left at room temperature for 1 hour. Also, glutathione was added to the polymer capsule solution having the disulfide group prepared according to Example 3 and then left at room temperature for one hour. In this case, an amount of glutathione was controlled such that the concentration of glutathione in the polymer capsule solution was 5 mM.

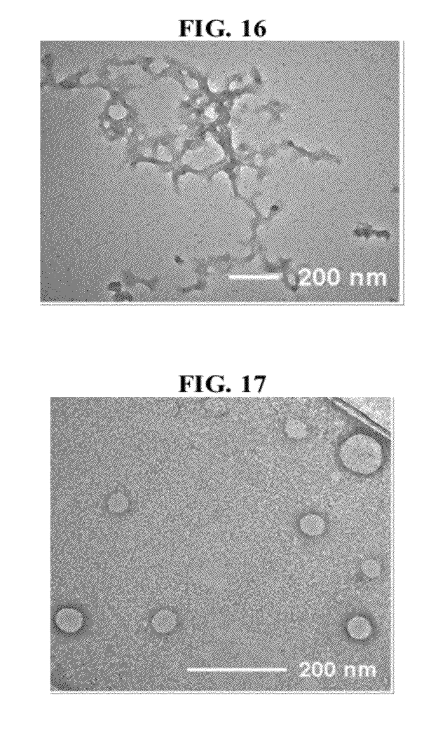

Then, the sample was observed via a transmission electron microscope and deformation of the polymer capsule was confirmed, which indicates that the polymer capsule had decomposed. Images of the transmission electron microscope are shown in FIG. 15 (the polymer capsule prepared according to Example 2) and FIG. 16 (the polymer capsule prepared according to Example 3). From these results, it was confirmed that when the polymer capsule is absorbed into a cell, under an acidic condition of a pH of 6.5 or less inside an endosome or under a reducing condition caused by glutathione inside a cell, the polymer capsule highly likely decomposes.

### Example 11

### Test for Confirming Decomposition of Polymer Capsule that Encapsulates 5(6)-Carboxyfluorescein and has Disulfide Group Under Glutathione Reducing Condition

3.4 mg (3-(2-aminoethanethio)propane-1-oxy) cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 1.1 mg of 5(6)-carboxyfluorescein were added thereto and dissolved. The result mixture was stirred for 24 hours at room temperature to form a polymer capsule. Then, reactants that were not polymerized or encapsulated were removed by dialysis to obtain a product solution.

One droplet of the product solution was dropped onto a planar substrate and dried, and a polymer capsule included therein was identified by using a transmission electron microscope. As a result, it was confirmed that a particle size of the polymer capsule was 90 nm and an image thereof is shown in FIG. 17.

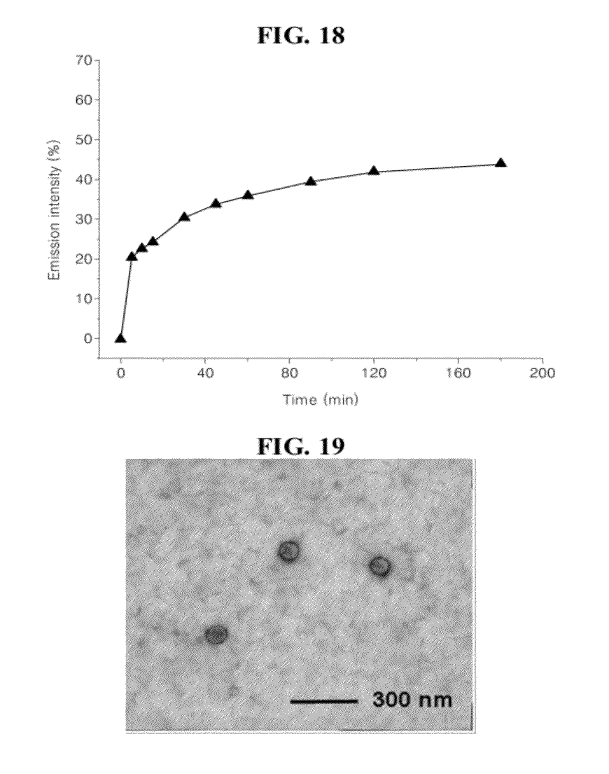

Then, glutathione was added to the product solution, and in this case, an amount of glutathione was controlled in such a manner that the concentration of glutathione in the product solution was 5 mM. Then, the fluorescence intensity of 5(6)-carboxyfluorescein was measured for 3 hours, and it was confirmed that the fluorescence intensity is increased over time. The fluorescence intensity results are shown in FIG. 18. The fluorescence intensity increases due to the fact that initially, fluorescence of 5(6)-carboxyfluorescein encapsulated in the polymer capsule having a disulfide group is self quenched, then once the polymer capsule is reduced by glutathione and decomposed, 5(6)-carboxyfluorescein is exposed. When one hour elapsed, a great amount of 5(6)-carboxyfluorescein was leaked from the polymer capsule. From these results, it was confirmed that a polymer capsule including a disulfide group well decomposes under reducing conditions.

### Example 12

### Test for Confirming that Polymer Capsule Encapsulating 5(6)-Carboxyfluorescein and Having a Disulfide Group and Including Folate-Sperimidine Enters a Cell by Endocytosis and the Polymer Capsule Decomposes in the Cell

3.4 mg (3-(2-aminoethanethio)propane-1-oxy) cucurbituril[6] was completely dissolved in 10 ml of a mixed solution including chloroform and methanol (a volumetric ratio of 5:5), and 50 μl of triethylamine, 2.4 mg of 3-[2-(2,5-dioxo-pyrrolidine-1-siloxycarbonyl)-ethyldisulfanyl]-propionic acid 2,5-dioxo-pyrrolidine-1-cyl ester, and 1.1 mg of 5(6)-carboxyfluorescein were added thereto and dissolved. The result mixture was stirred for 24 hours at room temperature to form a polymer capsule. 0.5 mg of folate-spermidine was added to the solution including the polymer capsule and then stirred for about 1 hour, and then the residual reactants that were not polymerized, encapsulated, or included were removed by dialysis. During the dialysis, in a final step, methanol was used as a solvent, thereby obtaining a product solution.

One droplet of the product solution was dropped onto a planar substrate and dried, and a polymer capsule included therein was identified by using a transmission electron microscope. As a result, it was confirmed that a particle size of the polymer capsule was 90 nm and an image thereof is shown in FIG. 19.

Then, 50 microliters of the refined polymer capsule dispersion was treated on KB cells which had been sufficiently cultured on RPMI-1640 medium (950 μl) with 5% CO₂ at a temperature of 37° C. Also, the entering of the polymer capsule was confirmed by using a con-focal laser scanning microscope. As a control, (a) KB cells, (b) KB cells treated with a polymer capsule that did not include folate-spermidine and encapsulated 5(6)-carboxyfluorescein, and (d) KB cells treated with a polymer capsule that included folate-spermidine, encapsulated 5(6)-carboxyfluorescein, and did not include a disulfide group were used. For reference, KB cells are representative oval cancer cells and include a great amount of a folate receptor at their surfaces. Accordingly, a polymer capsule having a surface reformed with folate may easily enter KB cells.

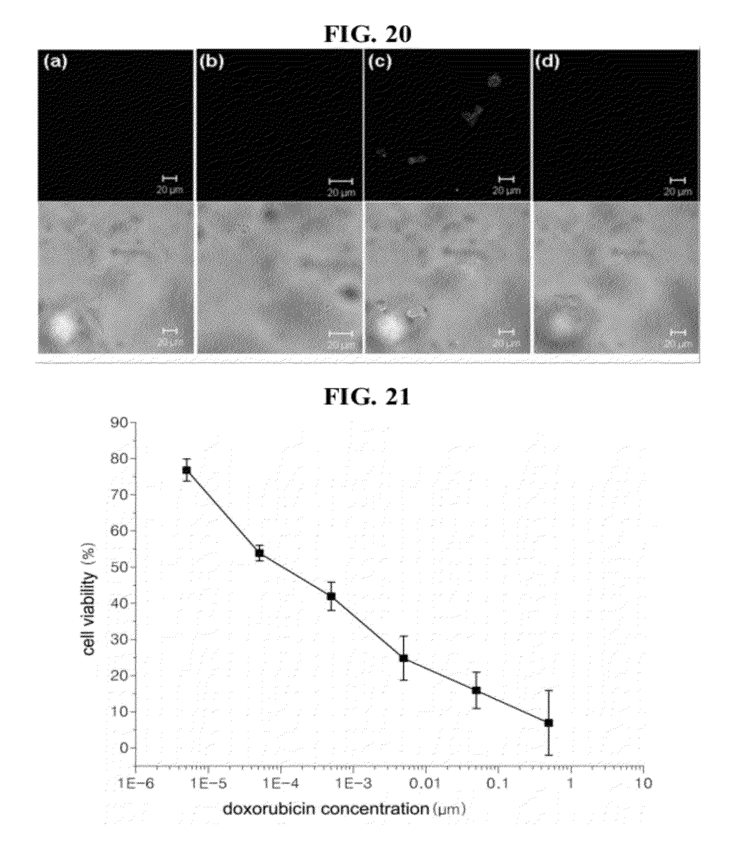

The results obtained using the con-focal laser scanning microscope are shown in FIG. 20. Referring to FIG. 20, it was confirmed that compared to the control, the polymer capsule including folate-spermidine and a disulfide group entered cells, and because the disulfide group was reduced by glutathione present in the cells, the polymer capsule decomposed, and thus fluorescence was well observed in the cells. From these results, it was confirmed that by reforming the surface of a polymer capsule with a cell-specific surface material, the polymer capsule is delivered target-specifically and a drug may be effectively delivered into cytoplasm.

### Example 13

### Test for Confirming Drug Delivery Efficiency of the Polymer Capsule that Encapsulates Doxorubicin and Includes Disulfide Group and Folate-Sperimidine, Prepared According to Example 9

KB cells were distributed at about 4000 cells/well on 96 wells and sufficiently cultured on RPMI-1640 medium 950 μl with 5% CO₂ at a temperature of 37° C. Then the KB cells were treated with 50 μl of the polymer capsule dispersion prepared according to Example 9 at various concentrations. The KB cells were more cultured for 60 hours, and viability of the cells was confirmed according to the concentration of the treated polymer capsule through a 3-(4,5-Dimethylthiazol-2-yl)-2,5-diphenyltetrazolium bromide, a tetrazole (MTT) test. When the KB cells were not treated with the polymer capsule, that is, in the case of the control, the cell viability was 99% or more. However, when the KB cells were treated with the polymer capsule encapsulating doxorubicin prepared according to Example 9, KB cells were effectively removed. The cell viability according to the doxorubicin concentration is shown in FIG. 21.

### INDUSTRIAL APPLICABILITY

A polymer capsule according to the present invention decomposes not in blood but in cells, and due to the inclusion of a pharmaceutically active material and/or target-specific material in the polymer capsule, a drug may be effectively delivered into a cytoplasm.

1. A polymer capsule manufactured by polymerizing a compound represented by Formula 1 below, or polymerizing the compound represented by Formula 1 below and a compound represented by Formula 2 below: 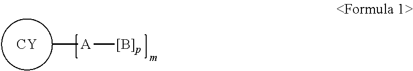
In Formula 1,
CY is a cucurbituril ring, a C2-C50 heteroaromatic ring, or a C6-C50 aromatic ring,
a plurality of A are each independently a chemical bond, or a C1 to 20 alkylene group, wherein one or more carbon atoms of the alkylene group are optionally substituted with one or more selected from the group consisting of —(C═O)—, —O(C═O)—, —O—, —S—, and —NH,
a plurality of B are each independently a C1 to 20 alkyl group, a C1 to 20 alkoxy group, a —C(═O)H, —COOH, —CH═CH₂, —C≡CH, —OH, or —NH₂,
-A-(B)p comprises one or more molecules other than carbon and hydrogen, and
p is an integer of 1 to 3, and m is an integer of 3 to 23, and
(Y₁)ⱼ—Z—(Y₂)ₖ <Formula 2>
In Formula 2,
Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,
Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and
j and k are each independently an integer of 1 to 3.

2. The polymer capsule of claim 1, wherein CY is a cucurbituril ring, a benzene ring, a naphthalene ring, an anthracene ring, a triphenylene ring, a pyrene ring, a coronin ring, a triazine ring, a phthalocyanine ring, a porphyrin ring, a pyridine ring, a quinoline ring, an anthraquinone ring, or a phenanthroline ring.

3. The polymer capsule of claim 1, wherein the compound represented by Formula 1 is represented by one of Formulae 3 to 10 below: 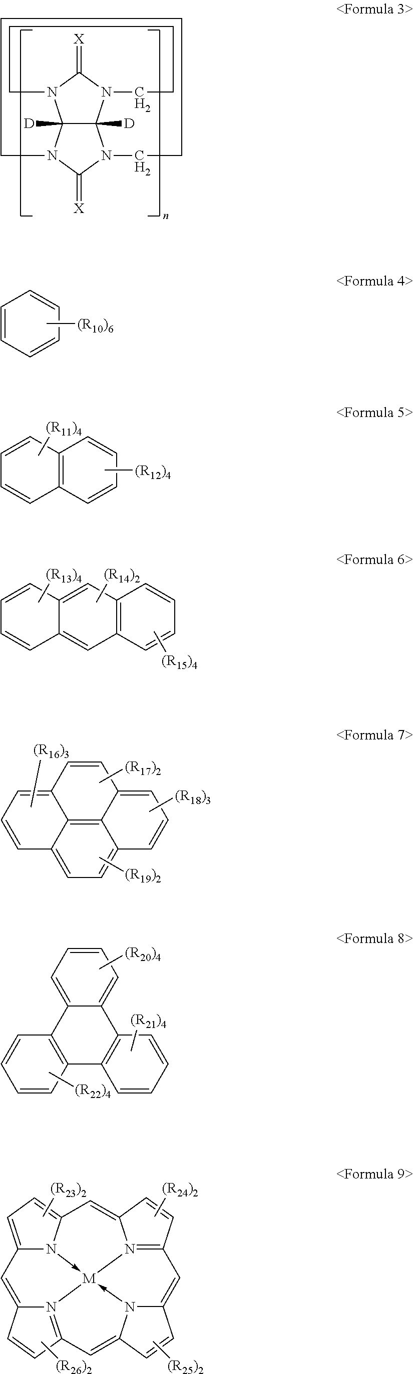 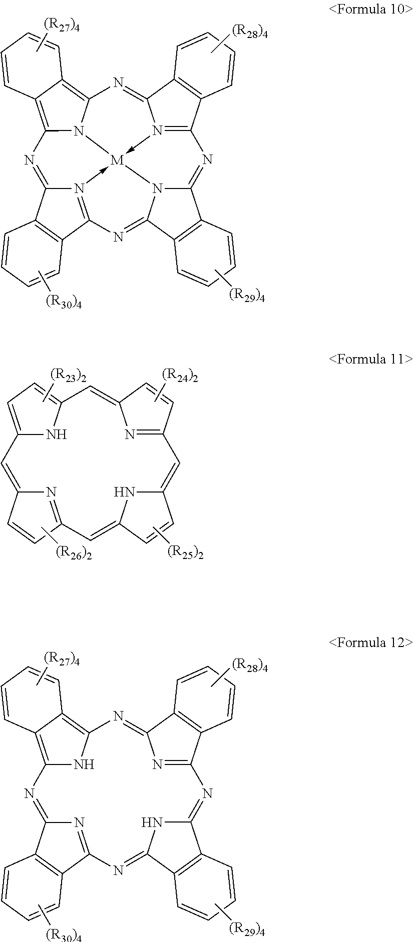
In the formulae above,
a plurality of D are each independently hydrogen or -A-(B)p, and 3 or more of D are -A-(B)p, a plurality of X are each independently O, S, or NH, and n is an integer of 4 to 20,
M is a metal,
R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, and R₃₀ are each independently hydrogen or -A-(B)p, and
3 or more of R₁₀, 3 or more selected from a plurality of R₁₁ and R₁₂, 3 or more selected from a plurality of R₁₃, R₁₄ and R₁₅, 3 or more selected from a plurality of R₁₆, R₁₇, R₁₈ and R₁₉, 3 or more selected from a plurality of R₂₀, R₂₁ and R₂₂, 3 or more selected from a plurality of R₂₃, R₂₄, R₂₅ and R₂₆, and 3 or more selected from a plurality of R₂₇, R₂₈, R₂₉ and R₃₀ are -A-(B)p.

4. The polymer capsule of claim 1, further comprising a target-specific compound included in an inner cavity of the cucurbituril ring.

5. The polymer capsule of claim 3, wherein the target-specific compound is represented by Formula 11 below:
E₁-G-E₂ <Formula 11>
In Formula 10,
G is a chemical bond, a C1 to 30 alkylene group, a C2 to 30 alkenylene group, a C2 to 30 alkynylene group, a C5 to 30 cycloalkylene group, a C6 to 30 arylene group, a C2 to 30 heteroarylene group, a C7 to 30 alkylarylene group, or a C7 to 30 arylalkylene group,
one or more carbon atoms of the alkylene group, alkenylene group, alkynylene group, cycloalkylene group, arylene group, or heteroarylene group are optionally substituted with one or more selected from the group consisting of —Si(Ra)(Rb)- (where Ra and Rb are each independently a C1 to 10 alkyl group), —(C═O)—, —O(C═O)—, —O—, —S—, and —NH—,
E₁ is a 1,3-diaminopropyl group, a 1,4-diaminobutyl group, a 1,5-diaminopentyl group, a 1,6-diaminohexyl group, a sperminyl group, a spermidinyl group, a propylamino group, a butylamino group, a pentylamino group, a hexylamino group, a biologinyl group, a pyridinyl group, a ferrocenyl group, or an amino acid group, and
E₂ is a radical of sugar, polypeptide, a protein, or a gene from which one hydrogen atom is removed, or a cation of sugar, polypeptide, a protein, or a gene from which one electron is removed.

6. The polymer capsule of claim 5, wherein the sugar is glucose, mannose, or galactose.

7. The polymer capsule of claim 5, wherein the protein is lectin, cellectin, or transferrin.

8. The polymer capsule of claim 4, wherein the target-specific compound is selected from the group consisting of folate-spermidine, glucose-sperminidine, mannose-sperminidine, galactose-sperminidine, lectin-spermine, cellectin-spermine, transferrin-spermine, and a combination thereof.

9. The polymer capsule of claim 1, further comprising a pharmaceutically active material encapsulated in the polymer capsule.

10. The polymer capsule of claim 9, wherein the pharmaceutically active material is selected from the group consisting of hydrocortisone, predsisolone, spironolactone, testosterone, megestrol acetate, danazol, progesterone, indomethacin, amphotericin B, or a combination thereof.

11. The polymer capsule of claim 9, wherein the pharmaceutically active material is selected from the group consisting of a human growth hormone, a granulocyte colony-stimulating factor (G-CSF), a granulocyte-macrophage colony-stimulating factor (GM-CSF), erythropoietin, vaccine, an antibody, insulin, glucagon, calcitonin, an adrenocorticotropic hormone (ACTH), somatostatin, somatotropin, somatomedin, a parathyroid hormone, a thyroid hormone, a hypothalamic secretion material, prolactin, endorphin, a vascular endothelial growth factor (VEGF), enkephalin, vasopressin, a nerve growth factor, a non-naturally occurring opioid, interferon, asparaginase, alginase, superoxide dismutase, trypsin, chymotrypsin, pepsin, or a combination thereof.

12. The polymer capsule of claim 1, wherein a diameter of the polymer capsule is in a range of 10 to 9000 nm.

13. A method of manufacturing a polymer capsule, the method comprising mixing a compound represented by Formula 1 below and a reaction catalyst to form a polymer capsule: 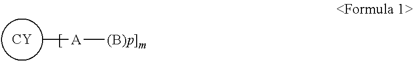
In Formula 1,
CY is a cucurbituril ring, a C2-C50 heteroaromatic ring, or a C6-C50 aromatic ring,
a plurality of A are each independently a chemical bond, or a C1 to 20 alkylene group, wherein one or more carbon atoms of the alkylene group are optionally substituted with one or more selected from the group consisting of —(C═O)—, —O(C═O)—, —O—, —S—, and —NH,
a plurality of B are each independently a C1 to 20 alkyl group, a C1 to 20 alkoxy group, —C(═O)H, —COOH, —CH═CH₂, —C≡CH, —OH, or —NH₂,
-A-(B)p comprises one or more molecules other than carbon and hydrogen, and
p is an integer of 1 to 3, and m is an integer of 3 to 23.

14. The method of claim 13, wherein the reaction catalyst is a Grubbs catalyst, an acidic catalyst, a basic catalyst, or a combination thereof.

15. The method of claim 14, wherein the acidic catalyst is para-toluene sulfonate, para-toluenesulfonyl chloride, HCl, H₂SO₄, HNO₃, or a combination thereof.

16. The method of claim 14, wherein the basic catalyst is N(CH₂CH₃)₃, pyridine, NaOH, NaBH₄, LiAlH₄, or a combination thereof.

17. A method of manufacturing a polymer capsule, the method comprising mixing a compound represented by Formula 1 below and a compound represented by Formula 2 below: 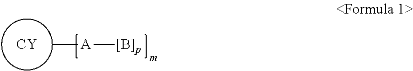
In Formula 1,
CY is a cucurbituril ring, a C2-C50 heteroaromatic ring, or a C6-C50 aromatic ring,
a plurality of A are each independently a chemical bond, or a C1 to 20 alkylene group, wherein one or more carbon atoms of the alkylene group are optionally substituted with one or more selected from the group consisting of —(C═O)—, —O(C═O)—, —O—, —S—, and —NH,
a plurality of B are each independently a C1 to 20 alkyl group, a C1 to 20 alkoxy group, —C(═O)H, —COOH, —CH═CH₂, —C≡CH, —OH, or —NH₂,
-A-(B)p comprises one or more molecules other than carbon and hydrogen, and
p is an integer of 1 to 3, and m is an integer of 3 to 23, and
(Y₁)ⱼ—Z—(Y₂)ₖ <Formula 2>
In Formula 2,
Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,
Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and
j and k are each independently an integer of 1 to 3.

18. The method of claim 13, wherein the polymer capsule is formed additionally using a pharmaceutically effective material:

19. The method of claim 18, wherein the pharmaceutically active material is selected from the group consisting of hydrocortisone, predsisolone, spironolactone, testosterone, megestrol acetate, danazol, progesterone, indomethacin, amphotericin B, and a combination thereof.

20. The method of claim 18, wherein the pharmaceutically active material is selected from the group consisting of a human growth hormone, a granulocyte colony-stimulating factor (G-CSF), a granulocyte-macrophage colony-stimulating factor (GM-CSF), erythropoietin, vaccine, an antibody, insulin, glucagon, calcitonin, an adrenocorticotropic hormone (ACTH), somatostatin, somatotropin, somatomedin, a parathyroid hormone, a thyroid hormone, a hypothalamic secretion material, prolactin, endorphin, a vascular endothelial growth factor (VEGF), enkephalin, vasopressin, a nerve growth factor, a non-naturally occurring opioid, interferon, asparaginase, alginase, superoxide dismutase, trypsin, chymotrypsin, pepsin, or a combination thereof.

21. The method of claim 18, further comprising mixing the polymer capsule encapsulating the pharmaceutically active material with a target-specific compound to include the target-specific compound in inner cavities of one or more cucurbituril rings that constitute the polymer capsule.

22. A method of manufacturing a polymer capsule, the method comprising:
mixing a compound represented by Formula 3 below, a compound represented by Formula 2 below, and a pharmaceutically active material to form a polymer capsule encapsulating the pharmaceutically active material; and
mixing the polymer capsule encapsulating the pharmaceutically active material with a target-specific compound to include the target-specific compound in inner cavities of one or more cucurbituril rings that constitute the polymer capsule. 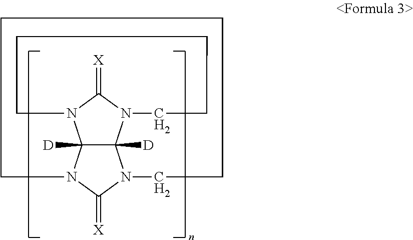
in Formula 3,
a plurality of D are each independently hydrogen or -A-(B)p,
a plurality of X are each independently O, S, or NH, n is an integer of 4 to 20,
from among D, 3 or more are -A-(B)p,
a plurality of A are each independently a chemical bond or a C1 to 20 alkylene group, wherein one or more carbon atoms of the alkylene group are optionally substituted with one or more selected from the group consisting of —(C═O)—, —O(C═O)—, —O—, —S—, and —NH—,
a plurality of B are each independently a C1 to 20 alkyl group, a C1 to 20 alkoxy group, —C(═O)H, —COOH, —CH═CH₂, —C≡CH, —OH, or —NH₂,
-A-(B)p comprises one or more molecules other than carbon and hydrogen, and
p is an integer of 1 to 3, and
(Y₁)ⱼ—Z—(Y₂)ₖ <Formula 2>
In Formula 2,
Z is a chemical bond, a C1 to 20 alkylene group, a C5 to 20 cycloalkylene group, a C5 to 20 arylene group, or a C2 to 20 heteroarylene group, wherein one or more carbon atoms of the alkylene group or cycloalkylene group are optionally substituted with one or more selected from the group consisting of —(R₁O)ᵣ— (where r is a real number of 1 to 10, and R₁ is a C1 to 5 alkylene group), —(C═O)—, —O(C═O)—, —O—, —S—, and —NH—,
Y₁ and Y₂ are each independently a C1 to 20 alkoxy group, a halogen group, a vinyloxy group, an N-acetoxysuccinimide group, —COOH, —N₃, —CH═CH₂, —C≡CH, —OH, or —NH₂, and
j and k are each independently an integer of 1 to 3.

23. The method of claim 22, wherein the pharmaceutically active material is selected from the group consisting of hydrocortisone, predsisolone, spironolactone, testosterone, megestrol acetate, danazol, progesterone, indomethacin, amphotericin B, or a combination thereof.

24. The method of claim 22, wherein the pharmaceutically active material is selected from the group consisting of a human growth hormone, a granulocyte colony-stimulating factor (G-CSF), a granulocyte-macrophage colony-stimulating factor (GM-CSF), erythropoietin, vaccine, an antibody, insulin, glucagon, calcitonin, an adrenocorticotropic hormone (ACTH), somatostatin, somatotropin, somatomedin, a parathyroid hormone, a thyroid hormone, a hypothalamic secretion material, prolactin, endorphin, a vascular endothelial growth factor (VEGF), enkephalin, vasopressin, a nerve growth factor, a non-naturally occurring opioid, interferon, asparaginase, alginase, superoxide dismutase, trypsin, chymotrypsin, pepsin, or a combination thereof.

25. The method of claim 22, wherein the target-specific compound is represented by Formula 11 below:
E₁-G-E₂ <Formula 11>
In Formula 10,
G is a chemical bond, a C1 to 30 alkylene group, a C2 to 30 alkenylene group, a C2 to 30 alkynylene group, a C5 to 30 cycloalkylene group, a C6 to 30 arylene group, a C2 to 30 heteroarylene group, a C7 to 30 alkylarylene group, or a C7 to 30 arylalkylene group,
one or more carbon atoms of the alkylene group, alkenylene group, alkynylene group, cycloalkylene group, arylene group, or heteroarylene group are optionally substituted with one or more selected from the group consisting of —Si(Ra)(Rb)- (where Ra and Rb are each independently a C1 to 10 alkyl group), —(C═O)—, —O(C═O)—, —O—, —S—, and —NH—,
E₁ is a 1,3-diaminopropyl group, a 1,4-diaminobutyl group, a 1,5-diaminopentyl group, a 1,6-diaminohexyl group, a sperminyl group, a spermidinyl group, a propylamino group, a butylamino group, a pentylamino group, a hexylamino group, a biologinyl group, a pyridinyl group, a ferrocenyl group, or an amino acid group, and
E₂ is a radical of sugar, polypeptide, a protein, or a gene from which one hydrogen atom is removed, or a cation of sugar, polypeptide, a protein, or a gene from which one electron is removed.
